(19) United States
(12) Patent Application Publication
(10) Pub. No.: US 2018/0015021 A1
(43) Pub. Date: Jan. 18, 2018

(54) HAIR CLEANSING RINSE

(71) Applicant: Henkel AG & Co. KGaA, Duesseldorf (DE)

(72) Inventors: Soeren Scheele, Hamburg (DE); Manuela Mette, Kleinfeld (DE); Thomas Schroeder, Hamburg (DE); Erik Schulze zur Wiesche, Hamburg (DE)

(73) Assignee: Henkel AG & Co. KGaA, Duesseldorf (DE)

(21) Appl. No.: 15/647,933

(22) Filed: Jul. 12, 2017

(30) Foreign Application Priority Data
Jul. 12, 2016 (DE) 10 2016 212 625.3

(51) Int. Cl. A61K 8/73; A61K 8/44; A61K 8/41; A61K 8/04; A61Q 5/02; A61K 8/65

(52) U.S. Cl. CPC A61K 8/732; A61Q 5/02; A61K 8/65; A61K 8/416; A61K 8/046; A61K 8/44; A61K 2800/30; A61K 2800/34

(57) ABSTRACT

Hair cleansing rinses which contain based on the weight of the total compositions:
a) from about 0.10 to about 30% by weight of at least one amphoteric surfactant,
b) from about 0.10 to about 15% by weight of at least one cationic surfactant of the quaternary ammonium compound type,
c) from about 0.01 to about 15% by weight of at least one hydrophobically modified starch compound and
d) from about 0.01 to about 2.00% by weight of at least one protein hydrolyzate have an excellent cleaning and conditioning effect.

Hair treated with these hair cleansing rinses has luster and resilience even after repeated use. There is no buildup effect on the hair. In addition, hair treated in this way has allows better distribution and rinse out than traditional hair conditioning compositions.

### CROSS-REFERENCE TO RELATED APPLICATION

This application claims priority to German Patent Application No. 10 2016 212 625.3, filed Jul. 12, 2016, which is incorporated herein by reference in its entirety.

### TECHNICAL FIELD

The present disclosure is in the field of cosmetics and relates to hair cleansing rinses containing a cationic surfactant and an amphoteric surfactant, a hydrophobically modified starch compound and a protein hydrolyzate. The present disclosure also relates to the use of the aforementioned combination of active ingredients for improving the skin tolerance of cosmetic cleaning compositions and for hair care.

### BACKGROUND

Traditional hair cleansing agents usually include anionic surfactants which are desired and necessary in order to be able to remove sebum and other types of soiling from the surface of hair and the scalp. However, these substances also cause the removal of lipids and proteins from the hair and/or the scalp during cleaning thereby necessitating a subsequent treatment with care agent to regenerate the hair.

In addition to the daily cleaning, however, the structure of hair may also be damaged due to environmental influences (such as intense sunlight exposure), mechanical stress (such as intense combing with the heat of a hair dryer), physical or chemical treatments (such as dyeing, perming or straightening hair). The consequences often include split ends, breakage of the hair and/or loss of luster of the hair. Such damaged hair is difficult to detangle and style.

There has therefore been a need for particularly mild hair cleansing agents which will care for the hair while at the same time removing as little of the oil as possible during the cleaning.

U.S. Pat. No. 6,723,309 proposes a novel form of hair care cleaning agent—so-called hair cleansing rinses. These products are based on a complex mixture of conditioners, cooling agents, emulsifiers, moisturizers, pH adjusting agents, preservatives, thickeners, herbal extracts and vitamins. These hair cleansing rinses clean the hair and impart more luster to the hair, giving it body and improved styling results. However, one disadvantage of these hair cleansing rinses is their low foaming power and poor distribution and/or rinse-out from hair. Furthermore, products that contain silicones result in an unwanted buildup effect on the hair when used regularly. This effect is especially pronounced in treatment of very fine and/or damaged hair.

### BRIEF SUMMARY

Hair cleansing rinses and cosmetic methods for treatment of hair are provided herein. In an embodiment, a hair cleansing rinse includes, based on the weight of the total composition:

a) from about 0.10 to about 30% by weight of at least one amphoteric surfactant,

b) from about 0.10 to about 15% by weight of at least one cationic surfactant of the quaternary ammonium compound type,

c) from about 0.01 to about 15% by weight of at least one hydrophobically modified starch compound and

d) from about 0.01 to about 2.00% by weight of at least one protein hydrolyzate.

In another embodiment, a cosmetic method for treatment of hair is provided. In accordance with the method, a hair cleansing rinse is applied to hair, massaged in and rinsed out after a treatment time. The hair cleansing rinse includes, based on the weight of the hair cleansing rinse:

a) from about 0.10 to about 30% by weight of at least one amphoteric surfactant,

b) from about 0.10 to about 15% by weight of at least one cationic surfactant of the quaternary ammonium compound type,

c) from about 0.01 to about 15% by weight of at least one hydrophobically modified starch compound and

d) from about 0.01 to about 2.00% by weight of at least one protein hydrolyzate.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

One object of the present disclosure is therefore to make available hair cleansing rinses which do not have the disadvantages defined above. In particular hair cleansing rinses that have a good foaming power and ideally do not contain any (sulfate) surfactants or any silicones and have a particularly mild cleansing effect are to be made available. At the same time, they should provide care for any type of hair (in particular brittle, damaged, dull, thin and/or stringy hair), so that it will have a pleasant feel, greater luster and resilience after being treated with the hair cleansing rinses.

It has now surprisingly been found that the objects defined above are achieved to an excellent extent by hair cleansing rinses which contain, in addition to a cationic surfactant and an amphoteric surfactant, a hydrophobically modified starch compound as well as a protein hydrolyzate.

Such hair cleansing rinses result in very good and protective cleaning products with a very good hair conditioning effect, which impart luster and resilience to hair even when used repeatedly and which do not result in a buildup effect. The hair cleansing rinses are therefore tolerated very well by skin.

Another advantage of the hair cleansing rinses is their excellent distribution on the hair as well as the fact that they can be rinsed out very well.

A first subject matter of this patent application is a hair cleansing product which contains—based on the weight of the total composition:
a) from about 0.10 to about 30% by weight of at least one amphoteric surfactant,
b) from about 0.10 to about 15% by weight of at least one cationic surfactant of the quaternary ammonium compound type,
c) from about 0.01 to about 15% by weight of at least one hydrophobically modified starch compound and
d) from about 0.01 to about 2.00% by weight of at least one protein hydrolyzate.

The hair cleansing rinses as contemplated herein preferably contain the active ingredients a) through d) in a suitable cosmetic vehicle. Within the scope of the present disclosure, this is preferably understood to be an aqueous vehicle, which preferably contains (based on the total weight of the hair cleansing rinses) at least about 55% by weight, more preferably at least about 60% by weight, especially preferably at least about 65% by weight and in particular preferably at least about 70% by weight water. In addition, the cosmetic vehicle may contain from about 0.01 to about 30% by weight, preferably from about 0.05 to about 20% by weight and in particular from about 0.1 to about 10% by weight of at least one alcohol.

Suitable alcohols include, for example, ethanol, ethyl diglycol, 1-propanol, 2-propanol, isopropanol, 1,2-propylene glycol, 1,3-propylene glycol, 1-butanol, 2-butanol, 1,2-butanediol, 1,3-butanediol, 1,4-butanediol, 1-pentanol, 2-pentanol, 1,2-pentanediol, 1,5-pentanediol, 1-hexanol, 2-hexanol, 1,2-hexanediol, 1,6-hexanediol, glycerin, polyethylene glycol, sorbitol, sorbitan, benzyl alcohol, phenoxyethanol or mixtures of these alcohols.

The water-soluble alcohols such as ethanol, isopropanol, 1,2-propylene glycol, glycerin, sorbitol, benzyl alcohol and/or phenoxyethanol as well as mixtures of these alcohols are preferred. In particular glycerin, isopropanol and/or 1,2-propylene glycol is/are especially preferred.

The hair cleansing rinses as contemplated herein contain as ingredient a) at least one amphoteric surfactant. A cleaning-active amount of amphoteric surfactant a), preferably mild, is necessary to achieve adequate quantities and qualities of foam. The term “amphoteric” surfactants is understood as contemplated herein to refer to amphoteric and/or zwitterionic surfactants.

Suitable amphoteric and/or zwitterionic surfactants a) in the sense of the present disclosure can preferably be selected from one or more compounds of the following formulas (I) to (VII) in which the radical R stands for a linear or branched, saturated or mono- or polyunsaturated alkyl or alkenyl radical with from about 7 to about 23 carbon atoms (formulas (I) and (II)) or for a linear or branched saturated or mono- or polyunsaturated alkyl or alkenyl radical with 8 to 24 carbon atoms (formulas (III) to (VII)):

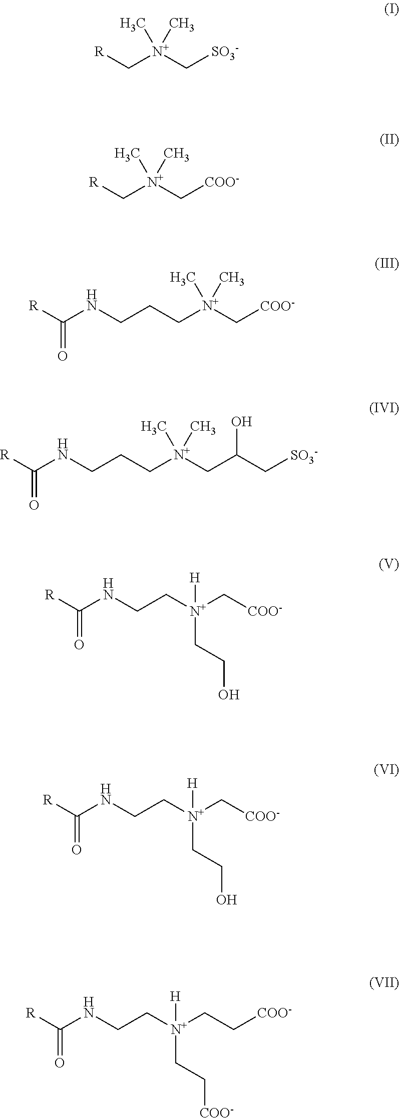

Amphoteric and/or zwitterionic surfactants of one of the aforementioned formulas (I) to (VII) preferably contain as the radical R predominantly a linear or branched saturated, mono- or polyunsaturated alkyl radical with from about 8 to about 20 carbon atoms, more preferably from about 8 to about 18 carbon atoms and in particular from about 8 to about 16 carbon atoms. Amphoteric and/or zwitterionic surfactants in which the radical R is derived from coconut fat are especially preferred. Most especially preferred are the amphoteric/zwitterionic surfactants that are known and commercially available from several providers under the INCI designations sodium cocoamphoacetate, disodium cocoamphodiacetate, sodium lauroamphoacetate, sodium lauroamphodiacetate, sodium cocoamphopropionate, disodium cocoamphodipropionate, coco betaine, lauryl betaine, cocamidopropyl betaine and/or lauramidopropyl betaine. Surfactants with the INCI designations cocamidopropyl betaine and/or cocoampho(di)acetate are preferred in particular.

The amount by weight of the at least one amphoteric surfactant a) in the total weight of the hair cleansing rinses as contemplated herein is preferably from about 0.50 to about 25.00% by weight, more preferably from about 0.75 to about 22.50% by weight, especially preferably from about 1.00 to about 20.00% by weight and in particular from about 1.50 to about 15.00% by weight. Especially preferably the hair cleansing rinses as contemplated herein contain amphoteric surfactants a) with the INCI designations cocamidopropyl betaine and/or cocoampho(di)acetate in the amounts specified previously.

The hair cleansing rinses as contemplated herein contain as ingredient b) at least one cationic surfactant of the quaternary ammonium compound type.

Preferred quaternary ammonium compounds include ammonium halides, in particular chlorides and bromides such as alkyl trimethyl ammonium chlorides, dialkyl dimethylammonium chlorides and trialkylmethylammonium chlorides, e.g., lauryl trimethyl ammonium chloride, cetyl trimethyl ammonium chloride, cetyl trimethyl ammonium bromide, cetyl trimethyl ammonium methosulfate, dicetyl dimethylammonium chloride, tricetylmethyl ammonium chloride, stearyl trimethyl ammonium chloride, distearyl dimethylammonium chloride, lauryl dimethyl benzylammonium chloride, behenyl trimethyl ammonium chloride, behenyl trimethyl ammonium bromide and/or behenyl trimethyl ammonium methosulfate as well as the imidazolium compounds known by the INCI designations quaternium-27 and quaternium-83.

The long alkyl chains of the aforementioned surfactants preferably have from about 10 to about 22 carbon atoms, more preferably from about 12 to about 22, especially preferably from about 14 to about 22 and in particular from about 16 to about 22. The compound known by the INCI designations behentrimonium chloride and/or cetrimonium chloride are particularly preferred.

The amount by weight of the at least one cationic surfactant b) in the total weight of the hair cleansing rinses as contemplated herein preferably amounts to from about 0.20 to about 12.50% by weight, more preferably from about 0.30 to about 10.00% by weight, especially preferably from about 0.40 to about 7.50% by weight and in particular from about 0.50 to about 5.00% by weight. The hair cleansing rinses as contemplated herein especially preferably contain cationic surfactants b) with the INCI designations cetrimonium chloride and/or behentrimonium chloride in the aforementioned amounts.

It has been found that an optimal balance between good foaming power and care can be achieved if the hair cleansing rinses as contemplated herein contain a combination of
a) cocoampho(di)acetate and/or cocamidopropyl betaine with
b) behentrimonium chloride and/or cetrimonium chloride in the aforementioned amounts.

The hair cleansing rinses as contemplated herein contain as ingredient c) at least one hydrophobically modified starch compound. This stabilizes the suds formed due to the surfactant combination a) and b) and imparts very good haptic properties to it. The suds are especially creamy and can be distributed excellently in hair. At the same time the addition of the hydrophobically modified starch compound c) results in an optimal thickening of the hair cleansing rinses as contemplated herein without causing them to be rigid and gelatinous.

Starch compounds that are suitable as contemplated herein are preferably those of plant origin and are preferably produced by starting with corn, wheat, rice, tapioca, potatoes and/or sago. The “hydrophobic modification” is understood as contemplated herein to refer to a chemical crosslinking, for example, by ionic crosslinking with calcium, aluminum and/or phosphates—preferably with phosphates—and/or chemical modification due to the introduction of hydrophobic groups. The term hydrophobic group is understood as contemplated herein to refer preferably to nonionic radicals such as hydroxyalkyl groups, for example, wherein “hydroxyalkyl groups” preferably denotes hydroxyethyl groups, hydroxypropyl groups and/or hydroxybutyl groups.

Starch compounds that are especially preferred as contemplated herein are crosslinked and have hydrophobic, nonionic substituents, especially preferably hydroxypropyl groups. The hydrophobically modified starch compounds known under the INCI designations hydroxyethyl starch phosphate and hydroxypropyl starch phosphate are most especially preferred. Hydroxypropyl starch phosphate is preferred in particular. The hydrophobically modified starch compounds c) are usually solids and preferably have average particle sizes in the range of 1 μm to 100 μm, more preferably of 5μm to 50 μm and in particular of 10 μm to 40 μm.

One example of a particularly preferred hydrophobically modified starch compound c) is the compound such as Structure® XL (Akzo Nobel), Structure® ZEA (National Starch) and Farinex® VA 70 (Avebe U.A.) which are known under the INCO designation hydroxypropyl starch phosphate and are available from several providers.

In a preferred embodiment, the at least one hydrophobically modified starch compound c) is therefore selected from starch phosphate compounds substituted with hydroxyalkyl groups, preferably from hydroxypropyl starch phosphates.

The amount by weight of the at least hydrophobically modified starch compound c) in the total weight of the hair cleansing rinses as contemplated herein is preferably from about 0.10 to about 12.50% by weight, preferably from about 0.25 to about 10.00% by weight and especially preferably from about 0.40 to about 7.50% by weight and in particular from about 0.50 to about 5.00% by weight. The hair cleansing rinses as contemplated herein especially preferably contain a hydrophobically modified starch compound c) having the INCI designation hydroxypropyl starch phosphate in the amounts specified previously.

In addition to good foaming properties and a particularly mild cleaning effect, the hair cleansing rinses as contemplated herein should also have an excellent care effect and should again impart more luster and resilience as well as an improved feel—in particular to thin and/or damaged hair.

It has been found that optimal care properties of the agents as contemplated herein can be achieved if at least one protein hydrolyzate d) is added as an additional care component to the agents as contemplated herein in addition to the cationic surfactant b) and the hydrophobically modified starch compound c). Protein hydrolyzates d) that are suitable as contemplated herein when used in combination with the cationic surfactant b) and the hydrophobically modified starch compound c) not only increase the luster and resilience of the hair but also improve the structure in particular for previously damaged hair and impart more smoothness and softness as well as a good feel to such hair.

Suitable protein hydrolyzates d) in the sense of the present disclosure are understood to include product mixtures that may be obtained by acidic, basic or enzymatically catalyzed degradation of proteins. Protein hydrolyzates of plant, animal and/or marine origin may be used. Animal protein hydrolyzates include, for example, elastin, collagen, keratin, silk and milk protein hydrolyzates, which may also be present in the form of salts. Such products are distributed under the brand names Dehylan® (Cognis), Promois® (Interorgana), Collapuron® (Cognis), Nutrilan® (Cognis), Gelitα-Sol® (Deutsche Gelatine Fabriken Stoess & Co), Lexein® (Inolex), ProSina® (Croda) and Kerasol® (Croda), for example. Suitable protein hydrolyzates of plant origin include, for example, soy, almond, rice, pea, potato and wheat protein hydrolyzates. Such products are available under the brand names Gluadin® (Cognis), DiaMin® (Diamalt), Lexein® (Inolex) and Crotein® (Croda), for example.

Cationized protein hydrolyzates, in which the underlying protein hydrolyzate may originate from an animal such as collagen, milk or keratin, from a plant, for example, wheat, corn, rice, potatoes, soy or almonds, for marine life forms, for example, fish collagen or algae or from protein hydrolyzates obtained by bioengineering, can be obtained from the corresponding proteins by a chemical hydrolysis, in particular alkaline or acidic hydrolysis, by enzymatic hydrolysis and/or by a combination of the two types of hydrolysis. The hydrolysis of proteins usually yields a protein hydrolyzate with a molecular weight distribution of approx. 100 Dalton up to several thousand Dalton. Such cationic protein hydrolyzates in which the protein fraction has a molecular weight of from about 100 to about 25,000 Dalton, preferably from about 250 to about 5000 Dalton, are preferred. In addition, cationic protein hydrolyzates are understood to include quaternated amino acids and mixtures thereof. Quaternation of protein hydrolyzates or amino acids is often carried out by employing quaternary ammonium salts such as N,N-dimethyl-N-(n-alky)-N-(2-hydroxy-3-chloro-n-propyl)ammonium halides, for example. In addition, the cationic protein hydrolyzates may also be further derivatized. Typical examples of cationic protein hydrolyzates and derivatives include the products that are known by the following INCI designations and are available commercially: cocodimonium hydroxypropyl hydrolyzed collagen, cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed silk, cocodimonium hydroxypropyl hydrolyzed soy protein, cocodimonium hydroxypropyl hydrolyzed wheat protein, cocodimonium hydroxypropyl silk amino acids, hydroxypropyl arginine lauryl/myristyl ether HCl, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratin, hydroxypropyltrimonium hydrolyzed rice bran protein, hydroxypropyltrimonium hydrolyzed silk, hydroxypropyltrimonium hydrolyzed soy protein, hydroxypropyl hydrolyzed vegetable protein, hydroxypropyltrimonium hydrolyzed wheat protein, hydroxypropyl trimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, lauryl dimonium hydroxypropyl hydrolyzed casein, lauryl dimonium hydroxypropyl hydrolyzed collagen, lauryl dimonium hydroxypropyl hydrolyzed keratin, lauryl dimonium hydroxypropyl hydrolyzed silk, lauryl dimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratin, steardimonium hydroxypropyl hydrolyzed rice protein, steardimonium hydroxypropyl hydrolyzed silk, steardimonium hydroxypropyl hydrolyzed soy protein, steardimonium hydroxypropyl hydrolyzed vegetable protein, steardimonium hydroxypropyl hydrolyzed wheat protein, steartrimonium hydroxyethyl hydrolyzed collagen, quaternium-76 hydrolyzed collagen, quatemium-79 hydrolyzed collagen, quatemium-79 hydrolyzed keratin, quaternium-79 hydrolyzed milk protein, quatemium-79 hydrolyzed silk, quatemium-79 hydrolyzed soy protein, quatemium-79 hydrolyzed wheat protein.

Particularly good care results can be achieved when the hair cleansing rinses as contemplated herein contain as protein hydrolyzate d) at least one protein hydrolyzate of animal origin, preferably a silk and/or keratin hydrolyzate (optionally cationically modified) and in particular a keratin hydrolyzate (optionally cationically modified) of one of the products obtainable under the brand names ProSina®, Nutrilan® keratin and/or Croquat® WKP PE LQ from the company Croda.

The amount by weight of the protein hydrolyzate(s) d) in the total weight of the hair cleansing rinses as contemplated herein preferably amounts to from about 0.03 to about 1.75% by weight, more preferably from about 0.05 to about 1.50% by weight, especially preferably from about 0.07 to about 1.25% by weight and in particular from about 0.10 to about 1.00% by weight. The hair cleansing rinses as contemplated herein especially preferably contain a keratin hydrolyzate (optionally cationically modified) in the aforementioned amounts.

The hair cleansing rinses as contemplated herein have an excellent care and/or moisturizing ability in addition to excellent foaming properties without having to add anionic surfactants and/or silicones to them. They have an excellent mildness and are suitable in particular for treatment of damaged hair or a sensitive dry scalp. Furthermore, they impart resilience and luster to hair without a buildup effect (even with regular use).

In another preferred embodiment, the hair cleansing rinses as contemplated herein are therefore essentially free of anionic surfactants. Within this embodiment it is particularly preferred if the hair cleansing rinses as contemplated herein are essentially free of anionic sulfate surfactants.

An even more preferred embodiment of the present disclosure is exemplified in that the hair cleansing rinses are essentially free of silicones.

The phrase “essentially free of ” is preferably understood to refer to an anionic (sulfate) surfactant content and/or silicone content of max. about 0.50% by weight, more preferably max. about 0.30% by weight, especially preferably max. about 0.10% by weight, and in particular 0% by weight, wherein the quantitative amounts are based on the total weight of the hair cleansing rinses as contemplated herein. The aforementioned maximum amounts apply to anionic (sulfate) surfactants that are free and/or can be added as aqueous solutions to the hair cleansing rinses as contemplated herein and/or silicones that are free and/or can be added as emulsions or dispersions but not for any amounts that may be present in subordinate amounts as secondary components in various commercial products.

For some embodiments it may be advantageous to add additional hair conditioning active ingredients e) to the hair cleansing rinses as contemplated herein to further increase and/or optimize the care properties. The optional additional care active ingredients e) must be easily incorporated into the hair cleansing rinses and must not have a negative influence on their stability properties and/or foaming properties. In addition, care must be taken to ensure that over-conditioning of hair, which could result in unwanted deposits on the hair and with regular use could lead to an unwanted buildup effect, must not occur due to the combination of the additional care substances) with the active ingredients a) through d).

Hair conditioning active ingredients e) that have proven to be especially advantageous include
vitamins (ei) and/or
oil, fat and/or wax components (eii).

Suitable oil, wax and/or fat components (eii) may preferably be selected from mineral and/or natural oil components and/or from fatty substances.

Usually triglycerides and mixtures of triglycerides are used as the natural (vegetable) oils. Preferred natural oils include coconut oil, (sweet) almond oil, walnut oil, peach kernel oil, apricot kernel oil, avocado oil, tea tree oil, soy oil, sesame oil, sunflower oil, tsubaki oil, evening primrose oil, rice bran oil, palm kernel oil, mango seed oil, meadowfoam seed oil, thistle oil, macadamia nut oil, grapeseed oil, amaranth seed oil, argan oil, bamboo oil, olive oil, wheat germ oil, pumpkin seed oil, malve oil, hazel nut oil, safflower oil, canola oil, sasanqua oil, jojoba oil, rambutan oil, cocoa butter and shea butter.

Mineral oils that may be used include in particular mineral oils, paraffin oils and isoparaffin oils as well as synthetic hydrocarbons. One example of a hydrocarbon that can be used is 1,3-di-(2-ethylhexyl)cyclohexane (Cetioll® S), which is available commercially. In addition, a dialkyl ether may be used as the oil component.

Dialkyl ethers that can be used include in particular di-n-alkyl ethers with a total of between about 12 and about 36 carbon atoms, in particular from about 12 to about 24 carbon atoms, such as, for example, di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert-butyl ether, diisopentyl ether, di-3-ethyldecyl ether, tert-butyl-n-octyl ether, isopentyl-n-octyl ether and 2-methylpentyl-n-octyl ether. The di-n-octyl ether that is available commercially under the brand name Cetioll® OE is especially preferred.

Fatty substances are understood to include fatty acids, fatty alcohols as well as natural and synthetic waxes, which may be present in an aqueous dispersion in either solid or in liquid form. Fatty acids that may be used include linear and/or branched, saturated and/or unsaturated fatty acid with 6 to 30 carbon atoms. Fatty acids with 10 to 22 carbon atoms are preferred. These include, for example, the isostearic acids such as the commercial products Emersol® 871 and Emersol® 875 as well as isopalmitic acids such as the commercial product Edenor® IP 95 as well as all other fatty acids distributed under the brand name Edenor® (Cognis). Additional typical examples of such fatty acids include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linoleic acid, eleostearic acid, arachic acid, gadoleic acid, behenic acid and erucic acid as well as technical mixtures thereof. The fatty acid cuts obtained from coconut oil or palm oil are especially preferred. Use of stearic acid is usually particularly preferred.

Fatty alcohols that may be used include saturated mono- or polyunsaturated branched or unbranched fatty alcohols with C₆-C₃₀, preferably C₁₀-C₂₂ and most especially preferably C₁₂-C₂₂ carbon atoms. For example, decanol, octanol, octenol, dodecenol, decenol, octadienol, dodecadienol, decadienol, oleyl alcohol, erucic alcohol, ricinoleic alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol can be used as well as their Guerbet alcohols. This list should serve as an example but should not be understood to be restrictive. However, the fatty alcohols are preferably derived from natural fatty acids, and production from the esters of fatty acids by reduction can be assumed in most cases. Fatty alcohol cuts created by reduction of naturally occurring triglycerides such as bovine tallow, palm oil, peanut oil, rapeseed oil, cottonseed oil, soy oil, sunflower oil and linseed oil or fatty acid esters obtained from their transesterification products with corresponding alcohols, which thus represent a mixture of different alcohols, may also be used as contemplated herein. Such substances are available commercially under the brand names Stenol®, for example, Stenol® 1618 or Lanette®, for example, Lanette® 0 or Lorol®, for example, Lorol® C8, Lorol® C14, Lorol® C18, Lorol® C8-18, HD-Ocenol®, Crodacol®, for example, Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is of course also possible to use as contemplated herein lanolin alcohols such as those available commercially under the brand names Corona®, White Swan®, Coronet® or Fluilant®.

Solid paraffins or isoparaffins, carnauba waxes, beeswaxes, candellila waxes, ozocerites, ceresin, spermaceti, sunflower wax, fruit waxes such as apple wax or citrus wax, microwaxes from PE or PP may also be used as the natural or synthetic waxes. Such waxes are available, for example, from Kahl & Co., Trittau. Additional solids include, for example:

Ester oils. Ester oils are understood to refer to the esters of C₆-C₃₀ fatty acids with C₂-C₃₀ fatty alcohols. The monoesters of fatty acids with alcohols having 2 to 24 carbon atoms are especially preferred. Examples of fatty acid component that may be used in the esters include caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachic acid, gadoleic acid, behenic acid and erucic acid as well as the technical grade mixtures thereof.

Examples of the fatty alcohol fraction in the ester oils include isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachyl alcohol, gadoleyl alcohol, behenyl alcohol, erucyl alcohol and brassidyl alcohol as well as the technical grade mixtures thereof. Especially preferred are isopropyl myristate (Rilanit® IPM), isononanoic acid C₁₆₋₁₈ alkyl ester (Cetioll® SN), 2-ethylhexylpalmitate (Cegesoft®24), stearic acid 2-ethylhexyl ester (Cetioll® 868), cetyl oleate, glycerol tricaprylate, coco fatty alcohol caprinate/caprylate (Cetioll® LC), n-butylstearate, oleyl erucate (Cetiol® J 600), isopropyl palmitate (Rilanit® IPP), oleyl oleate (Cetiol®), lauric acid hexyl ester (Cetiol® A), di-n-butyladipate (Cetiol® B), myristyl myristate (Cetiol® MM), cetearyl isononanoate (Cetiol® SN), oleic acid decyl ester (Cetiol® V),

Dicarboxylic acid esters such as di-n-butyladipate, di-(2-ethylhexyl)adipate, di-(2-ethylhexyl)succinate and diisotridecylacelate as well as diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate,

Symmetrical, asymmetrical or cyclic esters of carbonic acid with fatty alcohols,

Glycerol carbonate or dicaprylyl carbonate (Cetiol® CC),

Ethoxylated or non-ethoxylated mono-, di- and trifatty acid esters of saturated and/or unsaturated linear and/or branched fatty acids with glycerol, such as, for example, Monomuls® 90-O18, Monomuls® 90-L12, Cetiol® HE or Cutina® MD.

Especially preferred oil, wax and/or fat components (eii) in the sense of the present disclosure are selected from fatty alcohols and/or vegetable oils because of their care properties and properties that do not have a negative influence on the stability of the compositions as contemplated herein.

Examples of most especially preferred ingredient (eii) include cetyl alcohol, stearyl alcohol, cetearyl alcohol and/or one or more of the aforementioned vegetable oils.

The amount by weight of the oil, wax and/or fatty components (eii) in the total weight of the hair cleansing rinses as contemplated herein, preferably amounts to from about 0.01 to about 20.00% by weight, especially preferably from about 0.05 to about 15.00% by weight and in particular from about 0.10 to about 10.00% by weight.

In another preferred embodiment, the hair cleansing rinses as contemplated herein additionally contain, based on their weight, from about 0.10 to about 10.00% by weight, more preferably from about 0.25 to about 9.00% by weight, especially preferably from about 0.50 to about 8.00% by weight and in particular from about 0.75 to about 7.50% by weight of at least one saturated or unsaturated linear or branched C₁₂-C₂₄ alcohol, preferably selected from the compounds known by the INCI designations cetyl alcohol, stearyl alcohol and/or cetearyl alcohol.

Suitable vitamins (ei) preferably include the following vitamins, provitamins and vitamin precursors as well as their derivatives:

Vitamin A: the group of substances designated as vitamin A include retinol (vitamin A₁) and 3,4-didehydroretinol (vitamin A₂). The β-carotene is the provitamins of retinol. The vitamin A component may be considered to include, for example, vitamin A acid and its esters, vitamin A aldehyde and vitamin A alcohol as well as its esters and the palmitate and acetate.

Vitamin B: the following belong to the vitamin B group or the vitamin B complex:

vitamin B₁ (thiamine)

vitamin B₂ (riboflavin)

vitamin B₃. This designation is often used to refer to the compound nicotinic acid and nicotinamide (niacinamide).

vitamin B₅ (pantothenic acid and panthenol). Within the scope of this group, preferably panthenol is used. Derivatives of panthenol that can be used include in particular the esters and ethers of panthenol, pantolactone as well as cationically derivatized panthenols. Individual representatives include, for example, panthenol triacetate, panthenol monoethyl ether and its monoacetate as well as cationic panthenol derivatives.

vitamin B₆ (pyridoxine and pyridoxamine and pyridoxal).

Vitamin C (ascorbic acid): use in the form of the palmitic acid ester, the glucosides or phosphates may be preferred. Use in combination with tocopherols may also be preferred.

Vitamin E (tocopherols, in particular α-tocopherol).

Vitamin F: the term “vitamin F” is usually understood to refer to essential fatty acids, in particular linoleic acid, linolenic acid and arachidonic acid.

Vitamin H: vitamin H is understood to refer to the compound (3aS,4S,6aR)-2-oxohexahydrothienol[3,4-d]imidazole-4-valeric acid but in the meantime the trivial name biotin has been accepted for this substance.

Especially preferred are vitamins, provitamins and vitamin precursors (ei) from groups A, B, E and H; niacinamide, biotin, pantolactone and/or panthenol ae especially preferred. The amount by weight of the vitamin(s), vitamin derivative(s) and/or vitamin precursor(s) (ei) in the total weight of the hair cleansing rinses as contemplated herein preferably amounts to from about 0.001 to about 2.00% by weight, especially preferably from about 0.005 to about 1.00% by weight and in particular from about 0.01 to about 0.50% by weight.

In another preferred embodiment, the hair cleansing rinses as contemplated herein additionally contain—based on their weight—from about 0.001 to about 2.00% by weight, especially preferably from about 0.005 to about 1.00% by weight and in particular from about 0.01 to about 0.50% by weight of at least one vitamin, vitamin derivative and/or vitamin precursor, preferably selected from niacinamide and/or panthenol.

The hair cleansing rinses as contemplated herein are preferably traditional rinse-off products such as, for example, a hair care shampoo and/or a foaming hair care rinse that can be rinsed off. However, it is also possible and is preferable for some applications if the hair cleansing rinses as contemplated herein are applied as a hair cleansing foam. This ensures a particularly good distribution and dosability of the hair cleansing rinses.

Therefore, according to another particularly preferred embodiment, the hair cleansing rinses as contemplated herein are in the form of a cleaning hair care foam. The hair cleansing rinses as contemplated herein are therefore formulated in a dispensing device which represents either a pressurized gas container (aerosol container) filled with a propellant or a non-aerosol container. The pressurized gas containers with the help of which a product is distributed through a valve because of the internal gas pressure of the container is referred to by definition as an “aerosol container.” Conversely, a “non-aerosol container” is defined as a container under normal pressure, with the help of which a product is distributed by employing a mechanical action by a pump system or a squeeze system.

Within this embodiment, the hair cleansing rinses are in particular preferably in the form of an aerosol foam and an aerosol container. The hair cleansing rinses therefore preferably additionally contain at least one propellant.

Hair cleansing rinses as contemplated herein, which are in the form of an aerosol product, can be produced in the usual manner. As a rule all the ingredients of the hair cleansing rinses as contemplated herein except for the propellant are poured into a suitable pressure-resistant container. This is then closed with a valve. Finally the desired amount of propellant is added by traditional techniques.

In the embodiment as an aerosol foam, propellants that are suitable as contemplated herein are selected from N₂O, dimethyl ether, CO₂, air, alkanes with 3 to 5 carbon atoms, such as propane, n-butane, isobutane, n-pentane and isopentane and mixtures thereof, for example.

According to the embodiment of an aerosol foam, the aforementioned alkanes, mixtures of the aforementioned alkanes or mixtures of the aforementioned alkanes with dimethyl ether are used as the only propellant. However, the present disclosure explicitly also include concurrent use of propellants of the fluorochlorocarbon type, but in particular fluorocarbons. Dimethyl ether, propane, n-butane, isobutane and mixtures thereof are preferred.

Mixtures of propane and butane are most especially preferably used as the sole propellant in a weight ratio of propane to butane of from about 70:30 to about 15:85. These mixtures are in turn preferably used in the cosmetic hair cleansing rinses as contemplated herein in an amount of from about 1.00 to about 50.0% by weight, based on the weight of the total hair cleansing rinses. Amounts of from about 2.00 to about 40.0% by weight, especially preferably from about 3.00 to about 30.0% by weight and in particular from about 4.00 to about 20.0% by weight are especially preferred. Butane is understood as contemplated herein to refer to n-butane, isobutane and mixtures of n-butane and isobutane.

In another especially preferred embodiment, the hair cleansing rinse as contemplated herein is in the form of an aerosol foam in an aerosol container and additionally contains at least one propellant.

Within the third particularly preferred embodiment, it is especially advantageous if the hair cleansing rinses as contemplated herein contain the propellant, preferably a mixture of propane/butane, in an amount by weight of from about 1.00 to about 50.0% by weight, preferably from about 2.00 to about 40.0% by weight, especially preferably from about 3.00 to about 30.0% by weight and in particular from about 4.00 to about 20.0% by weight relative to the total weight of the composition.

Additional active ingredients, additives and auxiliary substances that may preferably be present in the hair cleansing rinses as contemplated herein include, for example:

nonionic surfactants, in particular alkyl (oligo)glycosides, amine oxides and/or fatty acid alkanolamides,

cationic polymers, in particular the compounds known by the INCI designations guar hydroxypropyltrimonium chloride, polyquaternium-10, polyquaternium-8, polyquaternium-7, polyquaternium-37, polyquaternium-67 and/or polyquaternium-87,

plant extracts,

moisturizers,

perfumes,

UV filters,

thickeners such as gelatins or vegetable gums, for example, agar, guar gum, alginates, xanthan gum, gum arabic, karaya gum, carob powder, linseed gum, dextrans, cellulose derivatives, for example, methyl cellulose, hydroxyalkyl cellulose and carboxymethyl cellulose, starch fractions and derivatives such as amylose, amylopectin and dextrins, clays and sheet silicates, such as bentonite or fully synthetic hydrocolloids such as polyvinyl alcohol, Ca, Mg or Zn soaps, for example,

structurants, such as maleic acid and lactic acid,

dimethylisosorbide,

cyclodextrins,

fiber structure-improving active ingredients, in particular mono-, di- and oligosaccharides such as, for example, glucose, galactose, fructose, fruit sugar and lactose, coloring agents for coloring the agent,

substances for adjusting the pH, for example, α- and β-hydroxycarboxylic acids such as citric acid, lactic acid, malic acid, glycolic acid,

active ingredients such as bisabolol and/or allantoin,

chelating agents such as EDTA, NTA, β-alanine diacetic acid and phosphonic acids,

ceramides. Ceramides are understood to be N-acylsphingosine (fatty acid amides of sphingosine) or synthetic analogs of such lipids (so-called pseudoceramides),

antioxidants,

preservatives such as sodium benzoate or salicylic acid,

additional viscosity regulating substances such as salts (NaCl).

Another subject matter of the present disclosure is the cosmetic use of
a) from about 0.10 to about 30% by weight of at least one amphoteric surfactant,
b) from about 0.10 to about 15% by weight of at least one cationic surfactant of the quaternary ammonium compound type,
c) from about 0.01 to about 15% by weight of at least one hydrophobically modified starch compound and
d) from about 0.01 to about 2.00% by weight of at least one protein hydrolyzate, in hair cleansing rinses to allow better distribution and/or rinse-out of the compositions.

A third subject matter of the present disclosure is the cosmetic use of a
a) from about 0.10 to about 30% by weight of at least one amphoteric surfactant,
b) from about 0.10 to about 15% by weight of at least one cationic surfactant of the quaternary ammonium compound type,
c) from about 0.01 to about 15% by weight of at least one hydrophobically modified starch compound and
d) from about 0.01 to about 2.00% by weight of at least one protein hydrolyzate, to improve the skin tolerance of cosmetic (hair) cleaning compositions.

A fourth subject matter of the present disclosure is the cosmetic use of the hair cleansing rinse as contemplated herein for mild cleaning and excellent care of hair and the scale, in particular brittle, damaged, dull, tin and/or stringy hair.

A fifth subject matter of the present disclosure is a cosmetic method for treatment of hair in which the hair cleansing rinse as contemplated herein is applied to hair, preferably wet, massaged in and then rinsed out after a treatment time.

For preferred embodiments of the compounds as contemplated herein and the method as contemplated herein, what was said above about the hair cleansing rinses as contemplated herein also applies here, mutatis mutandis.

The following examples should illustrate the subject matter of the present disclosure but without restricting it.

### EXAMPLE

### 1. Composition of Hair Cleansing Rinses as Contemplated Herein

The following hair cleansing rinses as contemplated herein were prepared, wherein all the numerical values in the following examples, unless otherwise indicated, corresponding to the amount of the respective active ingredient in % by weight, based on the total weight of the agent:

| | Formula 1 | Formula 2 | Formula 3 | Formula 4 | Formula 5 |
|---|---|---|---|---|---|
| Amphoteric surfactant | 0.10-30.00 | 0.50-25.00 | 0.75-22.50 | 1.00-20.00 | 1.50-15.00 |
| Cationic surfactant | 0.10-15.00 | 0.20-12.50 | 0.30-10.00 | 0.40-7.50 | 0.50-5.00 |
| (quaternary ammonium | | | | | |
| compound) | | | | | |
| Hydrophobically modified | 0.01-15.00 | 0.10-12.50 | 0.25-10.00 | 0.40-7.50 | 0.50-5.00 |
| starch compound | | | | | |
| Protein hydrolyzate | 0.01-2.00 | 0.03-1.75 | 0.05-1.50 | 0.07-1.25 | 0.10-1.00 |
| Water and optionally other | to 100 | to 100 | to 100 | to 100 | to 100 |
| additives and auxiliary | | | | | |
| substances | | | | | |
| | | | | | Formula |
| | Formula 6 | Formula 7 | Formula 8 | Formula 9 | 10 |
| Cocoampho(di)acetates | 0.10-30.00 | 0.50-25.00 | 0.75-22.50 | 1.00-20.00 | 1.50-15.00 |
| and/or cocoamidopropyl | | | | | |
| betaine | | | | | |
| Cationic surfactant | 0.10-15.00 | 0.20-12.50 | 0.30-10.00 | 0.40-7.50 | 0.50-5.00 |
| (quaternary ammonium | | | | | |
| compound) | | | | | |
| Hydrophobically modified | 0.01-15.00 | 0.10-12.50 | 0.25-10.00 | 0.40-7.50 | 0.50-5.00 |
| starch compound | | | | | |
| Protein hydrolyzate | 0.01-2.00 | 0.03-1.75 | 0.05-1.50 | 0.07-1.25 | 0.10-1.00 |
| Water and optionally other | to 100 | to 100 | to 100 | to 100 | to 100 |
| additives and auxiliary | | | | | |
| substances | | | | | |
| | Formula | Formula | Formula | Formula | Formula |
| | 11 | 12 | 13 | 14 | 15 |
| Cocoampho(di)acetates | 0.10-30.00 | 0.50-25.00 | 0.75-22.50 | 1.00-20.00 | 1.50-15.00 |
| and/or cocoamidopropyl | | | | | |
| betaine | | | | | |
| Behentrimonium chloride | 0.10-15.00 | 0.20-12.50 | 0.30-10.00 | 0.40-7.50 | 0.50-5.00 |
| and/or cetrimonium chloride | | | | | |
| Hydrophobically modified | 0.01-15.00 | 0.10-12.50 | 0.25-10.00 | 0.40-7.50 | 0.50-5.00 |
| starch compound | | | | | |
| Protein hydrolyzate | 0.01-2.00 | 0.03-1.75 | 0.05-1.50 | 0.07-1.25 | 0.10-1.00 |
| Water and optionally other | to 100 | to 100 | to 100 | to 100 | to 100 |
| additives and auxiliary | | | | | |
| substances | | | | | |
| | Formula | Formula | Formula | Formula | Formula |
| | 16 | 17 | 18 | 19 | 20 |
| Amphoteric surfactant | 0.10-30.00 | 0.50-25.00 | 0.75-22.50 | 1.00-20.00 | 1.50-15.00 |
| Cationic surfactant | 0.10-15.00 | 0.20-12.50 | 0.30-10.00 | 0.40-7.50 | 0.50-5.00 |
| (quaternary ammonium | | | | | |
| compound) | | | | | |
| Hydrophobically modified | 0.01-15.00 | 0.10-12.50 | 0.25-10.00 | 0.40-7.50 | 0.50-5.00 |
| starch compound | | | | | |
| Protein hydrolyzate | 0.01-2.00 | 0.03-1.75 | 0.05-1.50 | 0.07-1.25 | 0.10-1.00 |
| Water and optionally other | to 100 | to 100 | to 100 | to 100 | to 100 |
| additives and auxiliary | | | | | |
| substances | | | | | |
| | Formula | Formula | Formula | Formula | Formula |
| | 21 | 22 | 23 | 24 | 25 |
| Cocoampho(di)acetates | 0.10-30.00 | 0.50-25.00 | 0.75-22.50 | 1.00-20.00 | 1.50-15.00 |
| and/or cocoamidopropyl | | | | | |
| betaine | | | | | |
| Behentrimonium chloride | 0.10-15.00 | 0.20-12.50 | 0.30-10.00 | 0.40-7.50 | 0.50-5.00 |
| and/or cetrimonium chloride | | | | | |
| Hydrophobically modified | 0.01-15.00 | 0.10-12.50 | 0.25-10.00 | 0.40-7.50 | 0.50-5.00 |
| starch compound | | | | | |
| Protein hydrolyzate | 0.01-2.00 | 0.03-1.75 | 0.05-1.50 | 0.07-1.25 | 0.10-1.00 |
| Water and optionally other | to Σ 100 | to Σ 100 | to Σ 100 | to Σ 100 | to Σ 100 |
| additives and auxiliary | | | | | |
| substances | | | | | |
| | Formula | Formula | Formula | Formula | Formula |
| | 26 | 27 | 28 | 29 | 30 |
| Amphoteric surfactant | 0.10-30.00 | 0.50-25.00 | 0.75-22.50 | 1.00-20.00 | 1.50-15.00 |
| Cationic surfactant | 0.10-15.00 | 0.20-12.50 | 0.30-10.00 | 0.40-7.50 | 0.50-5.00 |
| (quaternary ammonium | | | | | |
| compound) | | | | | |
| Hydrophobically modified | 0.01-15.00 | 0.10-12.50 | 0.25-10.00 | 0.40-7.50 | 0.50-5.00 |
| starch compound | | | | | |
| Hydrolyzed keratin | 0.01-2.00 | 0.03-1.75 | 0.05-1.50 | 0.07-1.25 | 0.10-1.00 |
| Water and optionally other | to Σ 100 | to Σ 100 | to Σ 100 | to Σ 100 | to Σ 100 |
| additives and auxiliary | | | | | |
| substances | | | | | |
| | Formula | Formula | Formula | Formula | Formula |
| | 31 | 32 | 33 | 34 | 35 |
| Cocoampho(di)acetates | 0.10-30.00 | 0.50-25.00 | 0.75-22.50 | 1.00-20.00 | 1.50-15.00 |
| and/or cocoamidopropyl | | | | | |
| betaine | | | | | |
| Behentrimonium chloride | 0.10-15.00 | 0.20-12.50 | 0.30-10.00 | 0.40-7.50 | 0.50-5.00 |
| and/or cetrimonium chloride | | | | | |
| Hydrophobically modified | 0.01-15.00 | 0.10-12.50 | 0.25-10.00 | 0.40-7.50 | 0.50-5.00 |
| starch compound | | | | | |
| Hydrolyzed keratin | 0.01-2.00 | 0.03-1.75 | 0.05-1.50 | 0.07-1.25 | 0.10-1.00 |
| Water and optionally other | to Σ 100 | to Σ 100 | to Σ 100 | to Σ 100 | to Σ 100 |
| additives and auxiliary | | | | | |
| substances | | | | | |
| | Formula | Formula | Formula | Formula | Formula |
| | 36 | 37 | 38 | 39 | 40 |
| Cocoampho(di)acetates | 0.10-30.00 | 0.50-25.00 | 0.75-22.50 | 1.00-20.00 | 1.50-15.00 |
| and/or cocoamidopropyl | | | | | |
| betaine | | | | | |
| Behentrimonium chloride | 0.10-15.00 | 0.20-12.50 | 0.30-10.00 | 0.40-7.50 | 0.50-5.00 |
| and/or cetrimonium chloride | | | | | |
| Hydrophobically modified | 0.01-15.00 | 0.10-12.50 | 0.25-10.00 | 0.40-7.50 | 0.50-5.00 |
| starch compound | | | | | |
| Hydrolyzed keratin | 0.01-2.00 | 0.03-1.75 | 0.05-1.50 | 0.07-1.25 | 0.10-1.00 |
| Water and optionally other | to Σ 100 | to Σ 100 | to Σ 100 | to Σ 100 | to Σ 100 |
| additives and auxiliary | | | | | |
| substances | | | | | |
| | Formula | Formula | Formula | Formula | Formula |
| | 41 | 42 | 43 | 44 | 45 |
| Cocoampho(di)acetates | 0.10-30.00 | 0.50-25.00 | 0.75-22.50 | 1.00-20.00 | 1.50-15.00 |
| and/or cocoamidopropyl | | | | | |
| betaine | | | | | |
| Behentrimonium chloride | 0.10-15.00 | 0.20-12.50 | 0.30-10.00 | 0.40-7.50 | 0.50-5.00 |
| and/or cetrimonium chloride | | | | | |
| Hydrophobically modified | 0.01-15.00 | 0.10-12.50 | 0.25-10.00 | 0.40-7.50 | 0.50-5.00 |
| starch compound | | | | | |
| Hydrolyzed keratin | 0.01-2.00 | 0.03-1.75 | 0.05-1.50 | 0.07-1.25 | 0.10-1.00 |
| Propellant, in particular | 1.00-50.00 | 2.00-40.00 | 3.00-30.00 | 4.00-20.00 | 5.00-17.50 |
| propane/butane | | | | | |
| Water and optionally other | to Σ 100 | to Σ 100 | to Σ 100 | to Σ 100 | to Σ 100 |
| additives and auxiliary | | | | | |
| substances | | | | | |

| | | | Formula | Formula |
|---|---|---|---|---|
| | Formula 46 | Formula 47 | 48* | 49* |
| Sodium cocoamphoacetate | 8.00 | | | |
| Cocoamidopropyl betaine | | 10.00 | | |
| Behentrimonium chloride | 4.00 | | | |
| Cetrimonium chloride | | 3.00 | | |
| Hydroxypropyl starch phosphate | 3.00 | 3.50 | | |
| Hydrolyzed keratin | 0.20 | 0.30 | | 0.30 |
| Panthenol | 0.20 | | 0.20 | |
| Stearyl alcohol | | 6.00 | | 4.50 |
| Cetearyl alcohol | 5.00 | | 5.00 | |
| Citric acid | 1.80 | 2.00 | 0.30 | 0.70 |
| Lactic acid | | | 0.80 | 0.40 |
| Perfume | 0.80 | 0.60 | 0.50 | 0.60 |
| Stearamidopropyl dimethylamine | | | 2.00 | |
| Cocoamidopropyl dimethylamine | | | | 3.50 |
| Amodimethicone/morpholinomethyl | | | 0.25 | 0.50 |
| silsesquioxane copolymer | | | | |
| Amodimethicone | | | | 0.50 |
| Polyquaternium-37 | | | 0.20 | 0.50 |
| Ceteareth-20 | | | 0.50 | 0.40 |
| Preservative | q.s. | q.s. | q.s. | q.s. |
| Water | to Σ 100 | to Σ 100 | to Σ 100 | to Σ 100 |

*Examples not according to the present disclosure

### 2. Evaluation of the Skin Compatibility of Hair Cleansing Rinses as Contemplated Herein

100% healthy women (no allergies or skin problems) between the ages of 18 and 50 (50% 18 to 34 years old; 50% 35 to 50 years old) who had tested the hair cleansing rinses as contemplated herein in comparison with competing products and/or comparable products (in anonymized and coded form) in a blind test between Oct. 29 and Nov. 26, 2015 answered a questionnaire. Half of the women had used the product as contemplated herein three or four times a week and the other half used the comparative product.

The skin compatibility of the hair cleansing rinse of formula 46 as contemplated herein was evaluated in comparison with a traditional hair care shampoo (comprising as the surfactant 7% by weight (active substance) sodium lauryl ether sulfate as the surfactant instead of cocoamphoacetate and behentrimonium chloride and polyquaternium-10 as a care substance instead of hydroxypropyl starch phosphate and keratin hydrolyzate) with the help of a skin patch test.

The results of the patch test are summarized in the following Tables 1 and 2:

a) Treatment with the Cleaning Rinse According to Formula 41

TABLE 1

| | | Redness | | | | | | | | | | | | | | | |
|---|---|---|---|---|---|---|---|---|---|---|---|---|---|---|---|---|---|
| | | (erythema) | | | | Edema | | | | Flaking | | | | Cracking | | | |
| | No | Intensity | | | | | | | | | | | | | | | |
| Time | reaction | 1 | 2 | 3 | 4 | 1 | 2 | 3 | 4 | 1 | 2 | 3 | 4 | 1 | 2 | 3 | 4 |
| 6 hours | 16/20 | 4 | 1 | 0 | 0 | 0 | 0 | 0 | 0 | 0 | 0 | 0 | 0 | 0 | 0 | 0 | 0 |
| 24 hours | 17/20 | 2 | 0 | 0 | 0 | 0 | 0 | 0 | 0 | 1 | 0 | 0 | 0 | 0 | 0 | 0 | 0 |
| 48 hours | 18/20 | 0 | 0 | 0 | 0 | 0 | 0 | 0 | 0 | 2 | 0 | 0 | 0 | 0 | 0 | 0 | 0 |
| 72 hours | 16/10 | 0 | 0 | 0 | 0 | 0 | 0 | 0 | 0 | 4 | 0 | 0 | 0 | 0 | 0 | 0 | 0 |

b) Treatment with the Aforementioned Comparative Care Shampoo

TABLE 2

| | | Redness | | | | | | | | | | | | | | | |
|---|---|---|---|---|---|---|---|---|---|---|---|---|---|---|---|---|---|
| | | (erythema) | | | | Edema | | | | Flaking | | | | Cracking | | | |
| | No | Intensity | | | | | | | | | | | | | | | |
| Time | reaction | 1 | 2 | 3 | 4 | 1 | 2 | 3 | 4 | 1 | 2 | 3 | 4 | 1 | 2 | 3 | 4 |
| 6 hours | 8/20 | 6 | 6 | 0 | 0 | 0 | 0 | 0 | 0 | 0 | 0 | 0 | 0 | 0 | 0 | 0 | 0 |
| 24 hours | 5/20 | 6 | 7 | 2 | 0 | 0 | 0 | 0 | 0 | 0 | 0 | 0 | 0 | 0 | 0 | 0 | 0 |
| 48 hours | 5/20 | 9 | 4 | 0 | 0 | 0 | 0 | 0 | 0 | 1 | 1 | 0 | 0 | 0 | 0 | 0 | 0 |
| 72 hours | 7/20 | 6 | 0 | 0 | 0 | 0 | 0 | 0 | 0 | 7 | 0 | 0 | 0 | 0 | 0 | 0 | 0 |

It follows from the values in Tables 1 and 2 that the hair cleansing rinses as contemplated herein are tolerated better on skin than a traditional hair care shampoo based on anionic sulfate surfactants.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair cleansing rinse comprising, based on the weight of the total composition:
a) from about 0.10 to about 30% by weight of at least one amphoteric surfactant,
b) from about 0.10 to about 15% by weight of at least one cationic surfactant of the quaternary ammonium compound type,
c) from about 0.01 to about 15% by weight of at least one hydrophobically modified starch compound and
d) from about 0.01 to about 2.00% by weight of at least one protein hydrolyzate.

2. A hair cleansing rinse according to claim 1, comprising, based on its weight, from about 0.50 to about 25.00% by weight.

3. A hair cleansing rinse according to claim 1, comprising based on its weight, from about 0.20 to about 12.50% by weight of the at least one cationic surfactant b).

4. A hair cleansing rinse according to claim 1, wherein the at least one hydrophobically modified starch compound c) is selected from starch phosphate compound.

5. A hair cleansing rinse according to claim 1, comprising, based on its weight, from about 0.10 to about 12.50% by weight of the at least one hydrophobically modified starch compound c).

6. A hair cleansing rinse according to claim 1, wherein the at least one protein hydrolyzate d) is selected from protein hydrolyzates of animal origin.

7. A hair cleansing rinse according to claim 1, comprising based on its weight from about 0.03 to about 1.75% by weight of the at least one protein hydrolyzate d).

8. A hair cleansing rinse according to claim 1, essentially free of anionic sulfate surfactants.

9. A hair cleansing rinse according to claim 1, essentially free of silicones.

10. A hair cleansing rinse according to claim 1, further comprising, based on its weight, from about 0.10 to about 10.00% by weight of at least one saturated or unsaturated linear or branched C₁₂-C₂₄ alcohol.

11. A hair cleansing rinse according to claim 1, present as an aerosol foam in an aerosol container and additionally comprising at least one propellant in a weight amount of from 1.00 to about 50% by weight by weight of the total weight of the composition.

12. (canceled)

13. (canceled)

14. (canceled)

15. A cosmetic method for treatment of hair, in which a hair cleansing rinse is applied to hair, massaged in and rinsed out after a treatment time, wherein the hair cleansing rinse comprises, based on the weight of the hair cleansing rinse:
a) from about 0.10 to about 30% by weight of at least one amphoteric surfactant,
b) from about 0.10 to about 15% by weight of at least one cationic surfactant of the quaternary ammonium compound type,
c) from about 0.01 to about 15% by weight of at least one hydrophobically modified starch compound and
d) from about 0.01 to about 2.00% by weight of at least one protein hydrolyzate.

16. A hair cleansing rinse according to claim 1, present as an aerosol foam in an aerosol container and comprising, based on its weight,
from about 1.50 to about 15.00% by weight of the at least one amphoteric surfactant a),
from about 0.50 to about 5.00% by weight of the at least one cationic surfactant b),
from about 0.50 to about 5.00% by weight of the at least one hydrophobically modified starch compound c),
from about 0.10 to about 1.00% by weight of the at least one protein hydrolyzate d),
from 0.75 to about 7.50% by weight of at least one saturated or unsaturated linear or branched C₁₂-C₂₄ alcohol, and
from about 4.00 to about 20% by weight at least one propellant.

17. A hair cleansing rinse according to claim 2, wherein the at least one amphoteric surfactant a) is chosen from the group of compounds known by the INCI designations sodium cocoampho(di)acetate and/or cocamidopropyl betaine.

18. A hair cleansing rinse according to claim 1, wherein the at least one cationic surfactant b) is chosen from the group of compounds known by the INCI designations cetrimonium chloride and/or behentrimonium chloride.

19. A hair cleansing rinse according to claim 1, wherein the at least one hydrophobically modified starch compound c) is selected from hydroxypropyl starch phosphates substituted with hydroxyalkyl groups.

20. A hair cleansing rinse according to claim 1, wherein the at least one hydrophobically modified starch compound c) is a compound known by the INCI designation hydroxypropyl starch phosphate.

21. A hair cleansing rinse according to claim 1, wherein the at least one protein hydrolyzate d) is selected from silk and/or keratin hydrolyzates, optionally modified cationically.

22. A hair cleansing rinse according to claim 1, essentially free of anionic surfactant.

23. A hair cleansing rinse according to claim 10, wherein the at least one saturated or unsaturated linear or branched C₁₂-C₂₄ alcohol is selected from the compounds known by the INCI designations cetyl alcohol, stearyl alcohol and/or cetearyl alcohol.
